(19) United States
(12) Patent Application Publication
(10) Pub. No.: US 2006/0149303 A1
(43) Pub. Date: Jul. 6, 2006

(54) Acupuncture needle thermocouple clip for temperature and voltage output

(76) Inventor: Tien Huang, Arcadia, CA (US)

Correspondence Address: LAW OFFICES OF CLEMENT CHENG, 17220 NEWHOPE STREET #127, FOUNTAIN VALLEY, CA, 92708, US

(21) Appl. No.: 11/356,134

(22) Filed: Feb. 16, 2006

Related U.S. Application Data
(63) Continuation-in-part of application No. 11/026,961, filed on Dec. 30, 2004.

(51) Int. Cl. A61B 17/34

(52) U.S. Cl. 606/189

(57) ABSTRACT

An acupuncture needle has a staple having a pair of points for inserting into skin. The pair of points is connected at a bridge. A clip clips to the bridge and a pair of thermocouple metal members are mounted at opposing ends of the clip when the metal contact members contact an acupuncture needle when the pair of grasping ends are closed to grasp an acupuncture needle. The clip and a pair of grasping ends are shaped to grasp an acupuncture needle. The acupuncture needle forms a thermocouple junction.

This application is a continuation in part of co-pending application Ser. No. 11/026,961 to same inventor Huang, Tien Yu.

### DISCUSSION OF RELATED ART

Combining a variety of thermocouple metals can create a variety of thermocouples. Usual combinations are called types and identified by a letter such as B, C, E, J, K, N, R S, or T. The different types have different operating ranges and are typically used for different applications. The material of the metals used dictates the different applications. For example, type K has Chromel Alumel metals that generate voltage proportional to a difference in temperature pursuant to a Peltier-Seebeck effect. The voltage is then calibrated and analyzed to output a temperature reading.

Commonly, an infrared heat lamp can apply thermal radiation to a skin surface while acupuncture needles are applied to skin. The heat from the thermal radiation is easily measured because of the uniform temperature of the skin, and needles when an infrared heat lamp is used. When an infrared lamp is not used, the acupuncture needles are heated either by traditional moxabustion or by modern means such as electrical resistance heating. Localized heating presents difficult acupuncture needle temperature monitoring problems.

Measuring the temperature of acupuncture needles is difficult because the portion of the acupuncture needle entering the skin is small. Also, it is oftentimes desirable to measure the temperature of acupuncture needles while the acupuncture needles are in use. Also, sometimes only a voltage output is required.

Thermo couple switching is useful for monitoring temperature. A novel use of a thermocouple is shown in U.S. Pat. No. 4,821,285 describing the use of a thermocouple switch to monitor temperature of ore cars moving on rails. This shows that a wide range of thermocouple configurations can be used to output a measurable voltage.

The inventor previously disclosed a clip thermocouple device for measuring acupuncture needle temperature. The thermocouple clip device can be improved.

### BRIEF DESCRIPTION OF THE DRAWINGS

FIG. 1 is a side view of the clip.

FIG. 2 is a side exploded view of the clip.

FIG. 3 is a perspective view of the clip.

FIG. 4 is a perspective view of the clip holding an acupuncture needle and including the lead.

FIG. 5 shows a perspective view of the entire device including the clip, the lead, and the plug.

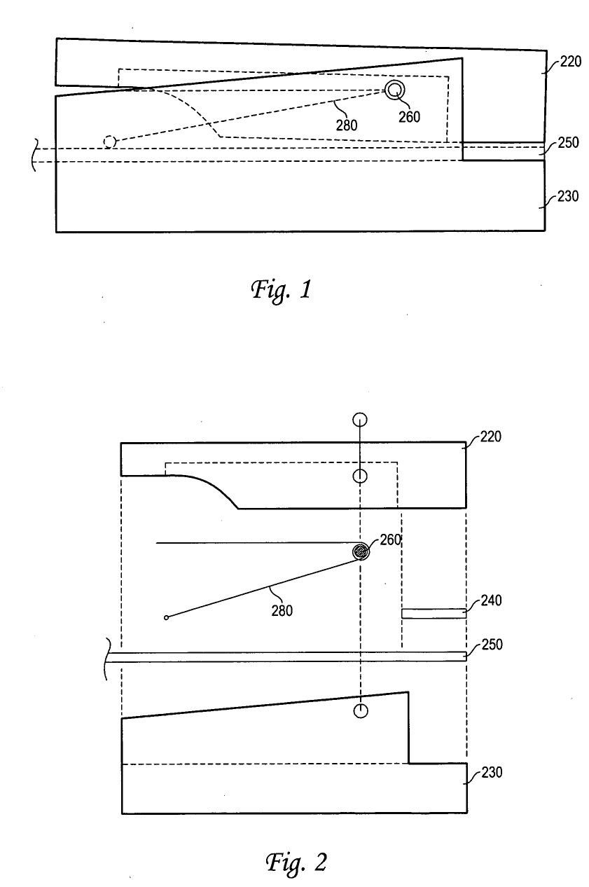

### DETAILED DESCRIPTION OF THE PREFERRED EMBODIMENT

The present device is used to monitor the voltage output of a thermocouple needle that can be used to deduce output temperature of an acupuncture needle, when the acupuncture needle is inserted into skin for treatment of a patient. The device includes a clip as shown in figure one. The clip has an upper member 220 and a lower member 230 mounted on a rotating axis 260. A spring member 280 formed as a coil spring is shown in figure one where the coil is retained by the axis 260 which is a pin formed as a small round rod that joins the upper and lower member and the spring. The spring force is enough to retain an acupuncture needle. The clip has wire mounted within. The electrical wire 250 carries voltage output signals from the thermocouple formed in the clip to a data logger thermometer or other such data analyzer for signal processing.

FIG. 2 shows an exploded view of the device. The top member 220 preferably has a hollow-section that nests with the lower member 230 and the axis pin 260 retains the members. The thermocouple has a pair of metal members 240 contacting an acupuncture needle when the clip attaches to an acupuncture needle. The clip can be manually opened to receive an acupuncture needle, and is biased closed by the spring.

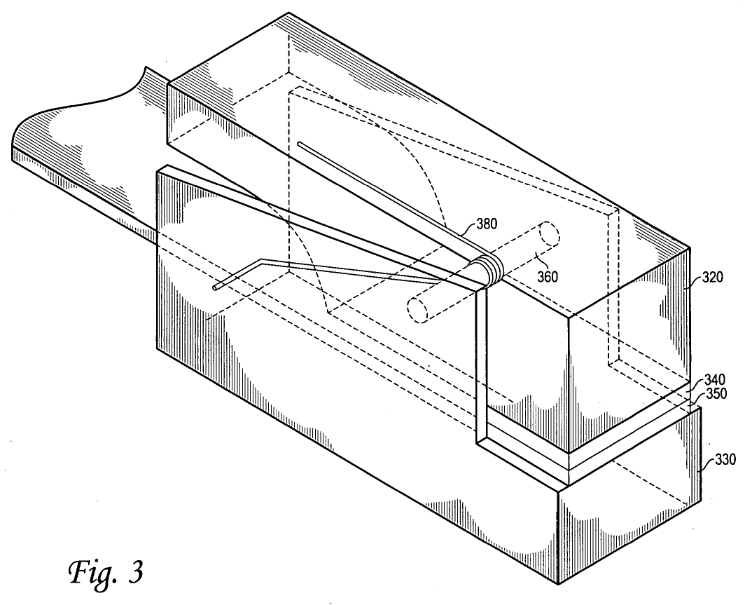

FIG. 3 shows a perspective view of a clip held in closed position by spring 380. The perspective view shows the nesting of the upper and lower members so that the upper 320 and lower member 330 move only along the rotational axis 360.

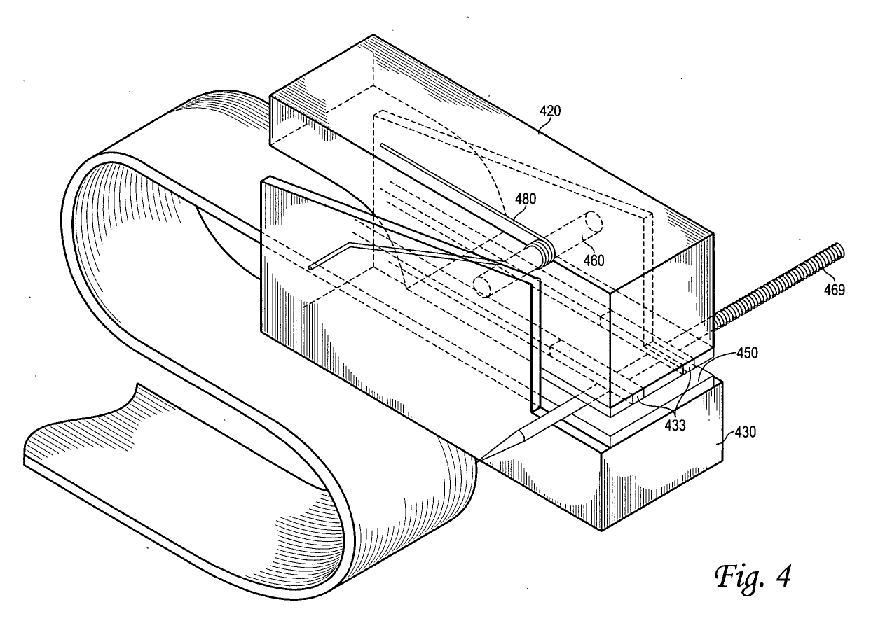

FIG. 4 shows the thermocouple clip having a pair of metal members 433 contacting the acupuncture needle 469 when the acupuncture needle 469 is held so the metal members 433 make electrical connection to the acupuncture needle. The acupuncture needle can be made of a variety of metals including stainless steel, gold, and silver. The acupuncture needle forms the thermocouple junction of the thermocouple sensor and becomes a part of the sensor. The metal members 433 are electrically connected to the wiring 450 that outputs a voltage. The voltage can be used in an electrical circuit with a data logger thermometer to calculate a temperature. The temperature is based upon electrical characteristics of the electrical circuit formed by the wiring 450, and the metal contact members 433. The other parts of the clip as seen in FIG. 4 include a spring 480, rotational axis 460 and the upper 420 and lower member 430. The perspective view shows the nesting configuration of the upper 420 and lower member 430 to limit motion in the direction parallel to the rotational axis 460.

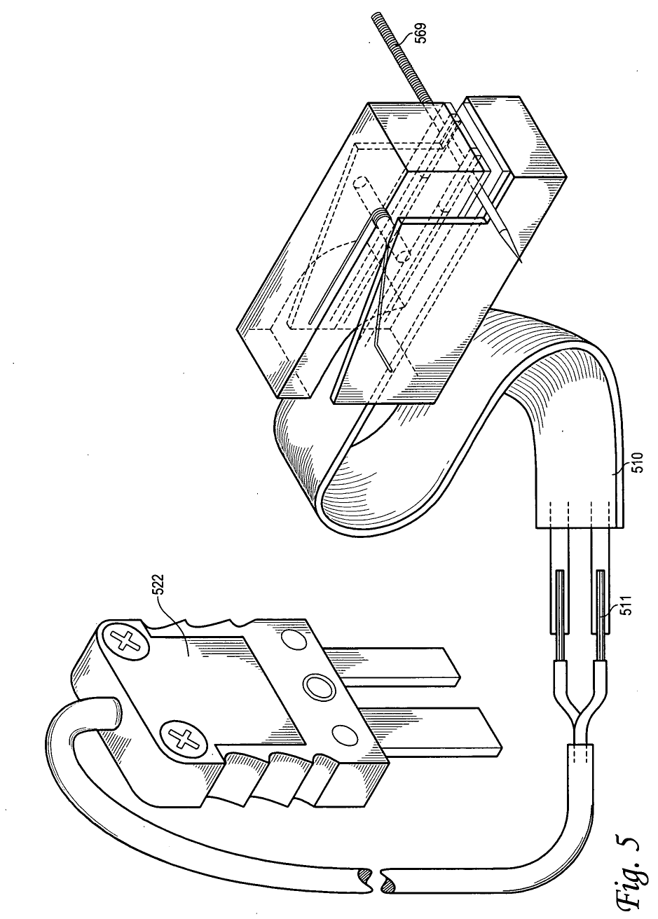

As seen in FIG. 5, the needle 569 can be held at a 90 degrees angle to the clip and the wiring 510 is initially a thin ribbon and can be joined a junction 511. The junction joins the thin ribbon wire with larger gauge wiring that continues to a plug 522 that plugs into an electrical device such as a data logger thermometer. The data logger thermometer is commonly available in the marketplace. The plug 522 is preferably of standard dimensions and specifications so that ordinary data logger thermometers may be used with the device. The plug 522 has a pair of prongs that have a voltage between them when the thermocouple is in use.

There are many needle embodiments that can work with the clip embodiments. For example, one can use a straight needle embodiment, and three embodiments having a staple shape needle. An alternative needle probe embodiment has a needle formed as in staple shape where leg upper ends are connected at a bridge. A needle can be made of a staple having a pair of points connected by a bridge. The needle can be inserted into the skin as an ordinary acupuncture needle would be inserted. The clip can then be attached to the dual prong staple. The clip would connect as previously described back to the data logger that interprets the voltage output and can use the voltage output to collect temperature information.

A second embodiment of the needle staple device is now described. The staple like needle alternatively can be made of a dissimilar thermocouple material, having a first leg and a second leg connected by the metal connector bridge. The first leg and second leg have a pair of points that are inserted into the skin. The first leg has a first thermocouple material that cooperates with the second thermocouple material comprising the second leg. The data logger has a pair of wires connecting to the first leg and second leg junction of the metal connector allowing the needle probe to capture the voltage of the system that can be translated into temperature information. The staple needle has connections that are integrally formed to the thermocouple material. Each leg preferably begins at a point and terminates at the connection that is connected to the metal connector.

As a third embodiment of the needle staple device, the wires leading to the upper ends of the legs connected to the junction can be made of dissimilar thermocouple materials. In this case, the different legs are made of the same material. The remainder of the configuration is as the second embodiment of the needle staple device. Accordingly, it is not intended that the invention be limited, except as by the appended claims.

### CALL OUT LIST OF ELEMENTS

220 Upper Clip Member
230 Lower Clip Member
240 Metal Contact
250 Wire Lead
260 Pivoting Axle
280 Spring Member
320 Upper Clip Member Of FIG. 3
330 Lower Clip Member Of FIG. 3
340 Metal Content Of FIG. 3
350 Wire Lead Of FIG. 3
360 Pivoting Axle Of FIG. 3
380 Spring Member Of FIG. 3
400 Clip
420 Upper Member Of FIG. 4
430 Lower Member Of FIG. 4
433 Metal Contacts Of FIG. 4
450 Wiring Of FIG. 4
460 Rotational Axis Of FIG. 4
469 Acupuncture Needle Of FIG. 4
480 Spring Of FIG. 4
510 Wire Lead Of FIG. 5
511 Junctions
522 Data Plug
569 Needle

1. An acupuncture needle comprising:
a. a staple having a pair of points for inserting into skin, wherein the pair of points are connected at a bridge;
b. a clip for clipping to the bridge;
c. a pair of thermocouple metal members mounted at opposing ends of the clip when the metal contact members contact an acupuncture needle when the pair of grasping ends are closed to grasp an acupuncture needle;
d. wherein the clip and a pair of grasping ends are shaped to grasp an acupuncture needle, whereby the acupuncture needle forms a thermocouple junction, wherein the having a voltage between them.

2. The acupuncture needle of claim 1, wherein the clip is formed of an upper member pivotally mounted to a lower number, the upper member forming an upper grasping end, and the lower member forming a lower grasping end.

3. The acupuncture needle of claim 2, wherein both metal contact members are formed on the upper member, whereby the acupuncture needle forms thermocouple junction when the pair of grasping ends close to hold the acupuncture needle.

4. The acupuncture needle of claim 1, wherein an upper grasping end formed on the pair of grasping ends has mounted on them the thermocouple metal members.

5. The acupuncture needle of claim 1, wherein the thermocouple metal members are mounted on an upper grasping end formed on the pair of grasping ends.

6. The acupuncture needle of claim 2, wherein the wires leading to the upper ends of the legs connected to the junction are made of dissimilar thermocouple materials and the legs are made of the same material.

7. The acupuncture needle of claim 2, wherein the wires leading to the upper ends of the legs connected to the junction are made of the same material and the legs are made of dissimilar thermocouple materials.

8. The acupuncture needle of claim 2, wherein the wires leading to the upper ends of the legs connected to the junction are made of dissimilar thermocouple materials and the legs are made of dissimilar thermocouple materials.

9. An acupuncture needle probe comprising:
a. a staple having two legs each having a point for inserting into skin, wherein the pair of legs form leg upper ends connected at a bridge;
b. a pair of thermocouple metal members formed on the staple;
c. wire leads connected to the upper ends of the legs; wherein the acupuncture needle forms a thermocouple junction, wherein a voltage varies between the wire leads according the variation of the staple temperature.

10. The acupuncture needle probe of claim 9, wherein the wires leading to the upper ends of the legs connected to the junction are made of dissimilar thermocouple materials and the legs are made of the same material.

11. The acupuncture needle probe of claim 9, wherein the wires leading to the upper ends of the legs connected to the junction are made of the same material and the legs are made of dissimilar thermocouple materials.

12. The acupuncture needle probe of claim 9, wherein the wires leading to the upper ends of the legs connected to the junction are made of dissimilar thermocouple materials and the legs are made of dissimilar thermocouple materials.

13. An acupuncture needle temperature monitoring device comprising:
a. a clip having a pair of grasping ends;
b. a pair of thermocouple metal members mounted at opposing ends of the clip when the metal contact members contact an acupuncture needle when the pair of grasping ends are closed to grasp an acupuncture needle; wherein the clip and a pair of grasping ends are shaped to grasp an acupuncture needle, whereby the acupuncture needle forms a thermocouple junction;
c. an electrical conductor electrically conducting the pair of thermocouple metal members to output a voltage.

14. The acupuncture needle of claim 13, wherein the needle is formed as a staple with a pair of points on a pair of legs connected by a bridge, wherein wires lead to the upper ends of the legs.

15. The acupuncture needle of claim 14, wherein the wires leading to the upper ends of the legs connected to the junction are made of dissimilar thermocouple materials and the legs are made of the same material.

16. The acupuncture needle of claim 14, wherein the wires leading to the upper ends of the legs connected to the junction are made of the same material and the legs are made of dissimilar thermocouple materials.

17. The acupuncture needle of claim 14, wherein the wires leading to the upper ends of the legs connected to the junction are made of dissimilar thermocouple materials and the legs are made of dissimilar thermocouple materials.
